(19) United States
(12) Patent Application Publication
(10) Pub. No.: US 2012/0184529 A1
(43) Pub. Date: Jul. 19, 2012

(54) COMBINATION THERAPY

(76) Inventors: Mark D. DeMario, New York, NY (US); Stanislaw M. Mikulski, Essex Fells, NJ (US)

(21) Appl. No.: 13/342,363

(22) Filed: Jan. 3, 2012

Related U.S. Application Data
(60) Provisional application No. 61/432,662, filed on Jan. 14, 2011.

(51) Int. Cl. A61K 31/55; A61P 35/00

(52) U.S. Cl. 514/212.04

(57) ABSTRACT

The present invention relates to a combination therapy of 2-Chloro-N-(4-chloro-pyridin-2-yl-phenyl)-4-methanesulfonyl-benzamide and 2,2-Dimethyl-N-((S)-6-oxo-6,7-dihydro-5H-dibenzo[b,d]azepin-7-yl)-N′-(2,2,3,3,3-pentafluoro-propyl)-malonamide for treating a patient suffering from a proliferative disorder.

This application claims the benefit of U.S. Provisional Application No. 61/432,662, filed Jan. 14, 2011, which is hereby incorporated by reference in its entirety.

### FIELD OF THE INVENTION

The present invention relates to a combination therapy for treating a patient suffering from a proliferative disorder, e.g., cancer.

### BACKGROUND OF THE INVENTION

Several studies have demonstrated an interaction between the Notch and Hh (hedgehog) signaling pathways in tumorigenesis and it is generally believed that the cross-talk between Notch and other signaling pathways plays important roles in cancer stem cells and tumor aggressiveness.

2-Chloro-N-(4-chloro-pyridin-2-yl-phenyl)-4-methanesulfonyl-benzamide (disclosed in Bioorg Med Chem Lett 19 (2009), pp. 5576-5581) (Compound A) is a small molecule antagonist of the Hh signaling pathway, with a molecular weight of approximately 421.30 g/mol. Specifically, Compound A binds to and inhibits SMO, blocking Hh signal transduction. In vitro and in vivo preclinical studies have demonstrated inhibition of Hh signaling following Compound A administration. Compound A has demonstrated efficacy against a variety of primary human tumor xenografts, including colorectal cancer (CRC) and pancreatic adenocarcinoma, and tumor cell-line xenograft models Inhibition of Hh signaling in xenograft models has been correlated with a decrease in tumor growth.

2,2-Dimethyl-N-((S)-6-oxo-6,7-dihydro-5H-dibenzo[b,d]azepin-7-yl)-N′-(2,2,3,3,3-pentafluoro-propyl)-malonamide (disclosed in WO 2005/023772 as useful for the treatment of Alzheimer's disease) (Compound B) is a water-soluble, orally-administered small molecule antagonist of γ-secretase, a key enzyme in the intramembrane proteolytic processing of several signaling receptors, including Notch, amyloid precursor protein (APP), CD44, and Her4. Blocking Notch signaling via γ-secretase inhibition produces a slower growing, less transformed phenotype in human cancer cells in vivo.

The Notch and Hedgehog signaling pathways, individually or combined, have been shown to be involved in many different tumors including breast cancer, colon cancer, and pancreatic cancer.

Combination of targeted agents which block multiple pathways involved in a given tumor's growth is a more attractive option and affords for more selectivity based on an individual's tumor mutation/genetic profile. Combination of multiple-targeted agents may also prove beneficial in reducing tumor stem cell proliferation following debulking of the primary tumor by cytotoxic±targeted therapy.

### SUMMARY OF THE INVENTION

The present invention provides a method of treating a patient suffering from a proliferative disorder, comprising administering to the patient: (A) a first component which comprises, as an active agent 2-Chloro-N-(4-chloro-pyridin-2-yl-phenyl)-4-methanesulfonyl-benzamide, or a pharmaceutically-acceptable salt thereof; and (B) a second component which comprises, as an active agent 2,2-dimethyl-N-((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof, the amounts of said active agents and dosing regimen being such that the combination thereof is therapeutically-effective in the treatment of said proliferative disorder, e.g., cancer.

The present invention also provides a kit comprising: (A) a first component which comprises, as an active agent 2-Chloro-N-(4-chloro-pyridin-2-yl-phenyl)-4-methanesulfonyl-benzamide, or a pharmaceutically-acceptable salt thereof; and (B) a second component which comprises, as an active agent 2,2-dimethyl-N-((S)-6-oxo-6,7-dihydro-5H-dibenzo[b,d]azepin-7-yl)-N′-(2,2,3,3,3-pentafluoro-propyl)-malonamide,

or a pharmaceutically-acceptable salt thereof.

The present invention further provides a composition comprising: (A) as an active agent 2-Chloro-N-(4-chloro-pyridin-2-yl-phenyl)-4-methanesulfonyl-benzamide, or a pharmaceutically-acceptable salt thereof; and (B) as an active agent 2,2-dimethyl-N-((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof.

In addition, the present invention provides a use of 2-chloro-N-(4-chloro-pyridin-2-yl-phenyl)-4-methanesulfonyl-benzamide, or a pharmaceutically-acceptable salt thereof; and 2,2-dimethyl-N-((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof, for the treatment of a proliferative disorder.

The present invention further provides a use of for the preparation of a medicament comprising 2-Chloro-N-(4-chloro-pyridin-2-yl-phenyl)-4-methanesulfonyl-benzamide, or a pharmaceutically-acceptable salt thereof and 2,2-dimethyl-N-((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof for the treatment of a proliferative disorder.

### DETAILED DESCRIPTION OF THE INVENTION

As used herein, the following terms have the meanings set out below.

As used herein, the term “Compound A” shall refer to 2-Chloro-N-(4-chloro-pyridin-2-yl-phenyl)-4-methanesulfonyl-benzamide. The compound has the structure shown below in formula I,

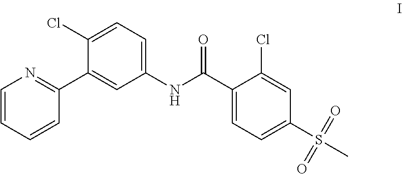

As used herein, the term “Compound I” shall refer to 2,2-dimethyl-N-((S)-6-oxo-6,7-dihydro-5H-dibenzo [b,d]azepin-7-yl)-N′-(2,2,3,3,3-pentafluoro-propyl)-malonamide. The compound has the structure shown below in formula I,

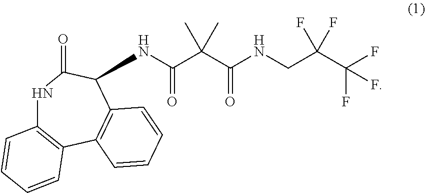

The term “antineoplastic” means inhibiting or preventing the development, maturation or proliferation of malignant cells.

The term “area under the curve” (AUC) is the area under the curve in a plot of concentration of drug in plasma against time. AUC represents the total amount of drug absorbed by the body, irrespective of the rate of absorption. This is useful for the therapeutic monitoring of drugs. Measurement of the drug concentrations in a patient's plasma and calculation of the AUC is useful to guide the dosage of this drug. AUC becomes useful for knowing the average concentration over a time interval, AUC/t. AUC is generally expressed as (mass*time/volume), for example, ng-hr/ml.

The term “pharmaceutically acceptable,” such as pharmaceutically acceptable carrier, excipient, etc., means pharmacologically acceptable and substantially non-toxic to the subject to which the particular compound is administered. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium, and quaternary ammonium hydroxides, such as for example, tetramethylammonium hydroxide.

The term “pharmaceutically acceptable ester” of a compound means a conventionally esterified compound having a carboxyl group, which esters retain the biological effectiveness and properties of the compound. Chemical modification of a pharmaceutical compound (i.e., drug) into a salt is a technique well known to pharmaceutical chemists to obtain improved physical and chemical stability, hydroscopicity, and solubility of compounds. See, e.g., H. Ansel et. al., Pharmaceutical Dosage Forms and Drug Delivery Systems (6^{th} Ed. 1995) at pp. 196 and 1456-1457.

The term “therapeutically-effective amount” means an amount of drug, which is effective for producing a desired therapeutic effect upon administration to a patient, for example, to stem the growth, or result in the shrinkage, of a cancerous tumor.

The term “therapeutic index” is an important parameter in the selection of anticancer agents for clinical trial. Therapeutic Index takes into consideration the efficacy, safety, pharmacokinetics, metabolism and bioavailability of anticancer agents. See, e.g., J. Natl. Cancer Inst. 81(13): 988-94 (Jul. 5, 1989).

The term “tumor control” means that the size of the tumor has either decreased, or has not increased by the defined and generally accepted criteria: e.g., the sum of the longest dimensions of the measurable tumor lesions has not increased by 20% or more as compared with the baseline, or with the shortest dimension of that lesion achieved post-treatment (RECIST=Response Evaluation Criteria in Solid Tumors, rules 1.1 published January 2009), or for specific tumor lesions such as those related to lymphoma and/or intracranial metastases of solid tumors, the sum of the products of the perpendicular diameters of measurable lesions has not increased by 25% or more from the baseline or from the last measurement. (See, e.g., World Health Organization (“WHO”) Handbook for Reporting Results of Cancer Treatment, Geneva (1979). In certain instances, the criteria for the volumetric (three-dimensional=3D) tumor measurements might be applied (e.g., for the brain metastatic lesions).

The present invention relates to a method of treating a patient suffering from a proliferative disorder, comprising administering to the patient: a first component which comprises, as an active agent, Compound A, or a pharmaceutically-acceptable salt thereof; and a second component which comprises, as an active agent, Compound B, or a pharmaceutically acceptable salt thereof, the amounts of said active agents being such that the combination thereof is therapeutically-effective in the treatment of said proliferative disorder.

The term “PK analysis” means the measurement of the extent and rate of absorption, distribution, metabolism and excretion of a drug given to a subject.
The term “dose limiting toxicities” means toxicities observed following the initial dose of Compounds A and B until the end of Cycle 2. Toxicities are graded using the descriptions and grading scales found in the revised NCI Common Terminology Criteria for Adverse Events (CTCAE) version 4.0.

Treatment of a proliferative disorder shall be understood to include maintaining or decreasing tumor size, inducing tumor regression (either partial or complete), inhibiting tumor growth, and/or increasing the life span of a patient suffering from said disorder.

In an embodiment of the present invention, the patient is a human.

In an embodiment of the invention, the proliferative disorder is a solid tumor, for example, breast cancer.

As stated above, each of the active agents administered in the aforementioned method are administered in amounts such that the combination thereof is therapeutically-effective in the treatment of said proliferative disorder.

The dosages may be administered according to any dosage schedule determined by the physician in accordance with the requirements of the patient. For example, the dosages of each of the two components may be administered in single or in divided doses over a period of several days, or alternating daily schedules.

In one embodiment (Schedule A), Compound B, or a pharmaceutically-acceptable salt thereof, is administered orally on day 1 at 5, 10 or 20 mg/day. Compound A, or a pharmaceutically-acceptable salt thereof is administered orally once daily at 150 mg. on day 8 and continuously thereafter for two weeks.

In a second embodiment (Schedule B), Compound B, or a pharmaceutically-acceptable salt thereof, is administered orally on day −2, −1 and 1 at 5, 10 or 20 mg/day (depending on dose limiting toxicities). Compound A, or a pharmaceutically-acceptable salt thereof is administered orally once daily at 150 mg. on day 8 and continuously thereafter for two weeks.

Subsequently patients on both Schedules A and B at the end of the initial 21 day schedule are treated with the combination of Compound A daily for an additional 21 days (at 150 mg/day and Compound B (at 5, 10 or 20 mg/day) on days 1-3 and 8-10 for 21 days.

The treatment cycle is repeated until disease progression or until development of significant toxicities.

Treatment with either agent may be sustained until a desired suppression of disease symptoms occurs or as long as the cancer remains under control.

### Example 1

CYCLE 1 (21 days): Compound B was administered once as a single agent orally (PO) at 5, 10 or 20 mg/day (depending on dose limiting toxicities)on Day 1 to allow for single-agent PK sampling. Compound A was administered as a single agent orally beginning on Day 8 and was given orally once a day on a continuous daily schedule as monotherapy for two weeks. Steady state PK analysis of Compound A occurred on Day 21.

SUBSEQUENT CYCLES (21 days): Administration of Compound A was dosed orally, once daily for 21 days. Administration of Compound B at 5, 10 or 20 mg/day was dosed orally on days 1-3, 8-10 every 21 days starting Day 22 (Cycle 2, Day 1) for a total of 6 days of Compound B administration per 21 day cycle.

During the first two cycles if administration of Compound B was well tolerated then the third week of both cycles which originally was to be a drug holiday is eliminated and Compound B is in addition administered on days 15-17 of each 21 day schedule.

### Example 2

CYCLE 1 (21 days): Compound B was administered as a single agent orally (PO) at 5, 10, or 20 mg/day (depending on dose limiting toxicities) on Day −2, −1, and 1. PK analysis of Compound B occurred on Day -2 and continued through Day 1. Compound A was administered as a single agent orally beginning on Day 8 and was given orally once daily on a continuous daily schedule as monotherapy for two weeks. Steady state PK analysis of Compound A occurred on Day 21.

SUBSEQUENT CYCLES (21 days): Administration of Compound A was dosed orally, once daily for 21 days. Administration of Compound B at 5, 10 or 20 mg/day was dosed orally on days 1-3, 8-10 every 21 days starting Day 22 (Cycle 2, Day 1) for a total of 6 days of Compound B administration per 21 day cycle.

During the first two cycles if administration of Compound B was well tolerated then the third week of both cycles which originally was to be a drug holiday is eliminated and Compound B is in addition administered on days 15-17 of each 21 day schedule.

What is claimed:

1. A method of treating a patient suffering from a proliferative disorder, comprising administering to the patient: (A) as an active agent, 2-Chloro-N-(4-chloro-pyridin-2-yl-phenyl)-4-methanesulfonyl-benzamide, or a pharmaceutically-acceptable salt thereof, and (B) as an active agent, 2,2-dimethyl-N-((S)-6-oxo-6,7-dihydro-5H-dibenzo[b,d]azepin-7-yl)-N′-(2,2,3,3,3-pentafluoro-propyl)-malonamide, or a pharmaceutically acceptable salt thereof, the amounts of said active agents being such that the combination thereof is therapeutically-effective in the treatment of said proliferative disorder.

2. A method according to claim 1 wherein said proliferative disorder is a solid tumor.

3. A method according to anyone of claim 1 or 2 wherein said proliferative disorder is a breast tumor.

4. A method according to anyone of claims 1-3 wherein the 2-Chloro-N-(4-chloro-pyridin-2-yl-phenyl)-4-methanesulfonyl-benzamide is dosed at 150 mg/day.

5. A method according to anyone of claims 1-4 wherein the 2,2-dimethyl-N-((S)-6-oxo-6,7-dihydro-5H-dibenzo[b,d]azepin-7-yl)-N′-(2,2,3,3,3-pentafluoro-propyl)-malonamide is provided at a dose selected from 5, 10 or 20 mg/day.

6. A method according to any one of claims 1 to 5 wherein 2,2-dimethyl-N-((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof, is administered once daily on day 1 of a 21 day cycle in an amount of about 5 mg and subsequently on days 1-3 and 8-10 of a second 21 day cycle.

7. A method according to any one of claims 1 to 5 wherein 2,2-dimethyl-N-((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof, is administered once daily on day 1 of a 21 day cycle in an amount of about 10 mg and subsequently on days 1-3 and 8-10 of a second 21 day cycle.

8. A method according to any one of claims 1 to 5 wherein 2,2-dimethyl-N-S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof, is administered once daily on day 1 of a 21 day cycle in an amount of about 20 mg and subsequently on days 1-3 and 8-10 of a second 21 day cycle.

9. A method according to claim any one of claims 6 to 8 wherein 2-Chloro-N-(4-chloro-pyridin-2-yl-phenyl)-4-methanesulfonyl-benzamide is administered in an amount of about 150 mg on days 8-21 of the first 21 day cycle and subsequently once daily during the second 21 day cycle.
